(19) United States
(12) Patent Application Publication
(10) Pub. No.: US 2024/0374412 A1
(43) Pub. Date: Nov. 14, 2024

(54) UNDERGARMENT FOR HOLDING AN OSTOMY BAG

(71) Applicant: Jenell Currence, Phoenix, AZ (US)

(72) Inventor: Jenell Currence, Phoenix, AZ (US)

(21) Appl. No.: 18/144,670

(22) Filed: May 8, 2023

(51) Int. Cl. A61F 5/449

(52) U.S. Cl. CPC A61F 5/449

(57) ABSTRACT

A clothing article for holding an ostomy bag in a selected location on an undergarment includes an undergarment is configured to be worn over a lower torso and genitals of the user. The undergarment has a waistband which extends around the torso of the user when the undergarment is worn by the user, and a pouch is mounted to an outer surface of the waistband. The pouch is configured for holding the ostomy bag and comprising a wall bounding a cavity for at least partially containing the ostomy bag. The pouch is removably attached to the waistband.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to ostomy bag holding garments and more particularly pertains to a new ostomy bag holding garment for holding an ostomy bag in a selected location on an undergarment.

### (2) Description of Related Art including information disclosed under 37 CFR 1.97 and 1.98

The prior art describes several devices for attaching an ostomy bag to a user's person, including attaching the ostomy bag to an undergarment worn by the user. However, the prior art does not disclose such a device in which a pouch for holding the ostomy bag is selectively positionable in each of a plurality of locations on a waistband of the undergarment, which facilitates the positioning of the ostomy bag in a comfortable location on the user's body near a stoma.

### BRIEF SUMMARY OF THE INVENTION

An embodiment of the disclosure meets the needs presented above by generally comprising an undergarment is configured to be worn over a lower torso and genitals of the user. The undergarment has a waistband which extends around the torso of the user when the undergarment is worn by the user, and a pouch is mounted to an outer surface of the waistband. The pouch is configured for holding the ostomy bag and comprising a wall bounding a cavity for at least partially containing the ostomy bag. The pouch is removably attached to the waistband.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top front side perspective view of a clothing article according to a first embodiment of the disclosure.

FIG. 2 is an exploded view of the first embodiment of the disclosure.

FIG. 3 is a front view of the first embodiment of the disclosure.

FIG. 4 is a top view of the first embodiment of the disclosure.

FIG. 5 is a front view of a second embodiment of the disclosure.

FIG. 6 is a side view of the second embodiment of the disclosure.

FIG. 7 is a bottom view of the second embodiment of the disclosure.

FIG. 8 is a front view of a third embodiment of the disclosure.

FIG. 9 is a side view of the third embodiment of the disclosure.

FIG. 10 is a bottom view of the third embodiment of the disclosure.

FIG. 11 is an in-use view of the first embodiment of the disclosure.

### DETAILED DESCRIPTION OF THE INVENTION

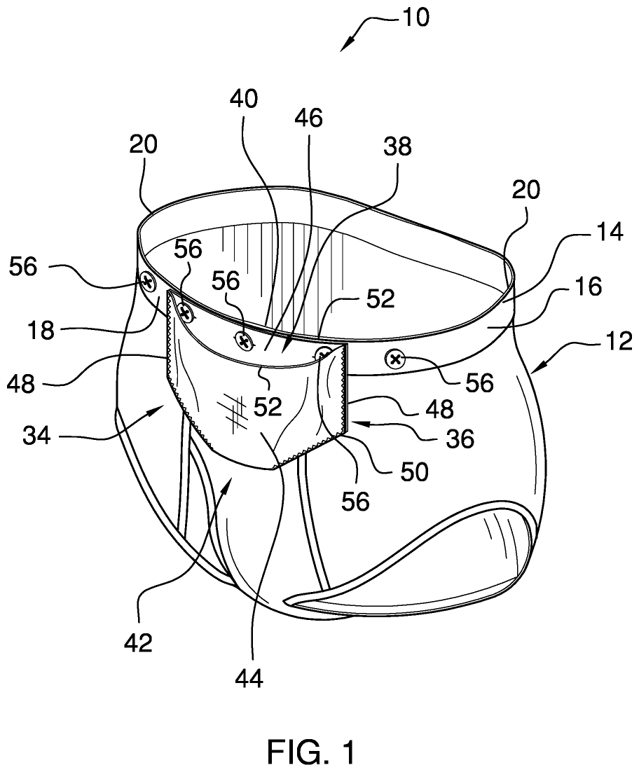

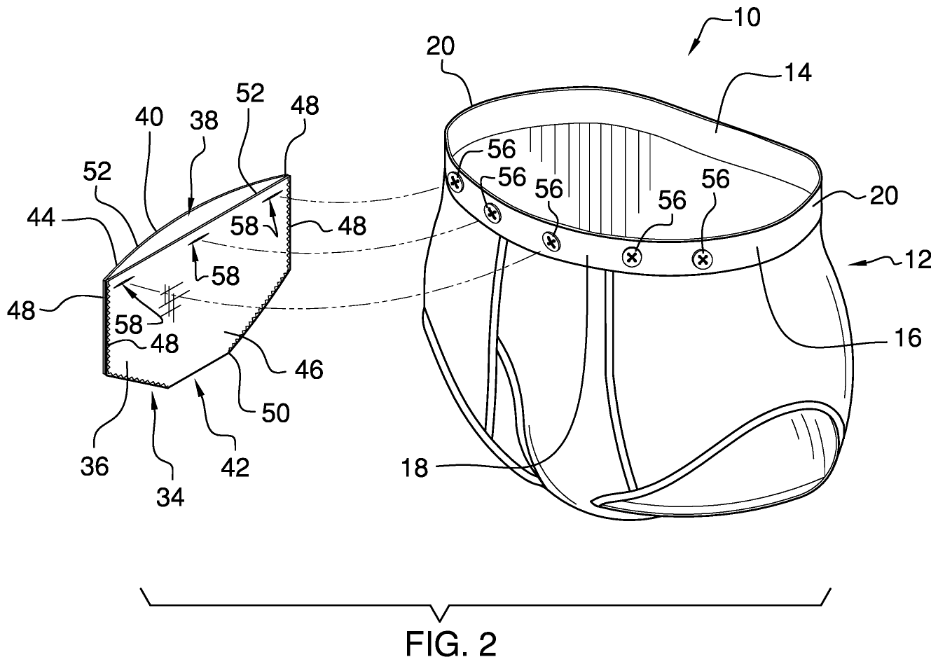

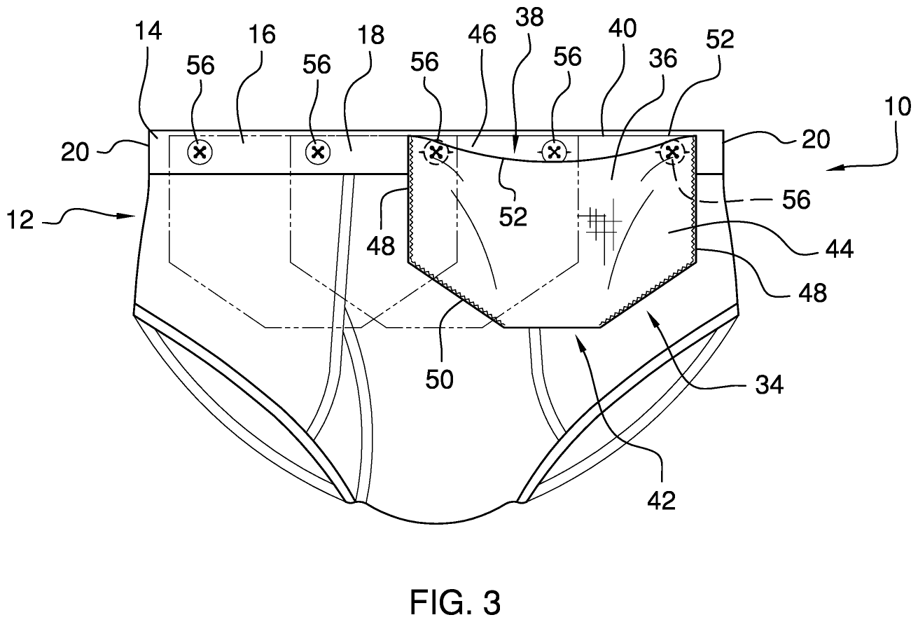

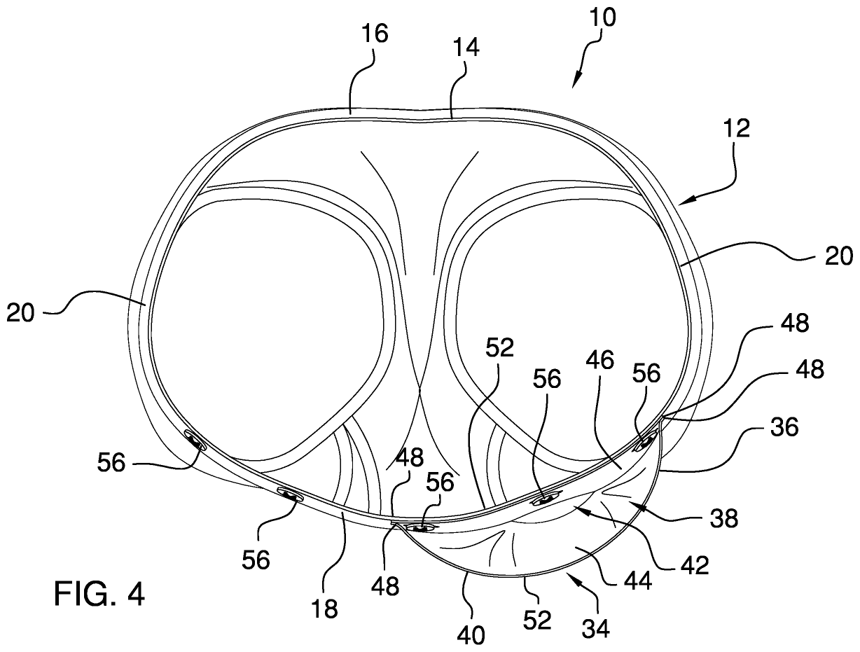

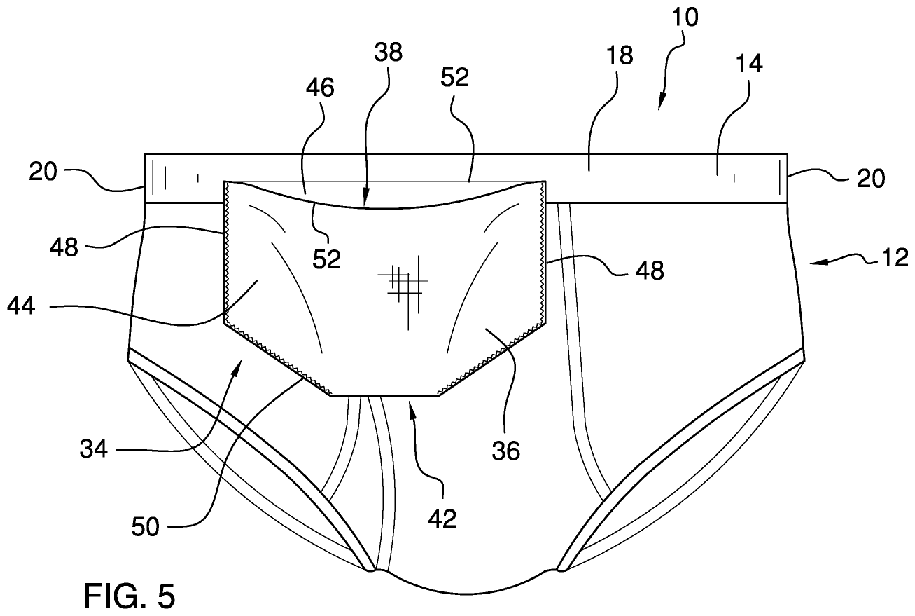

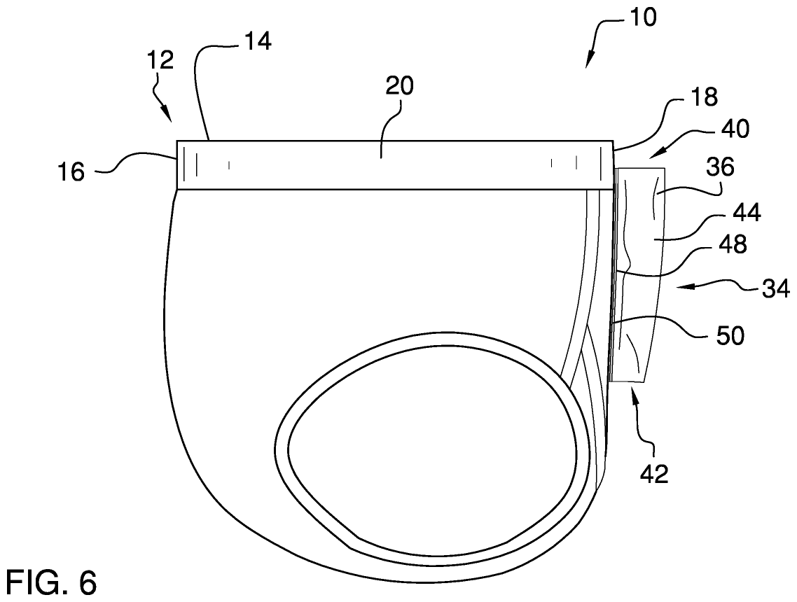

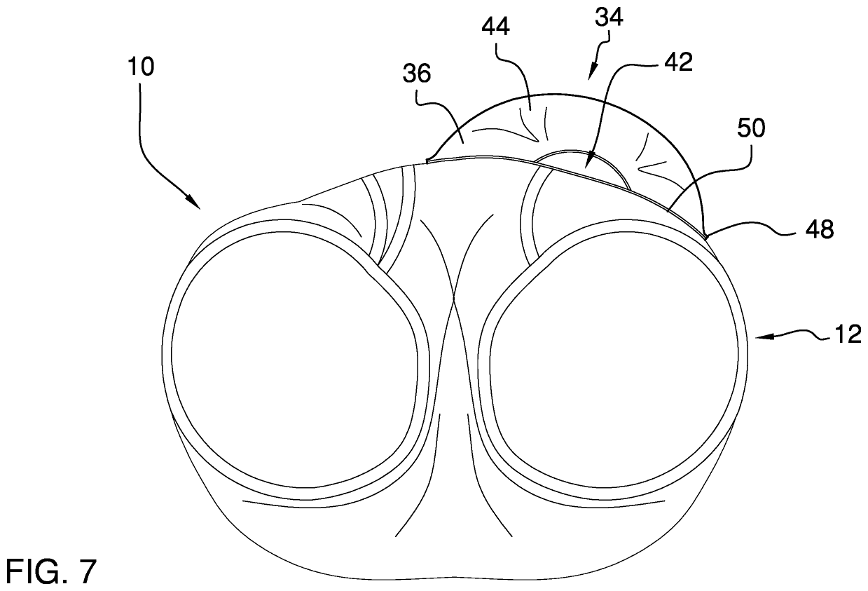

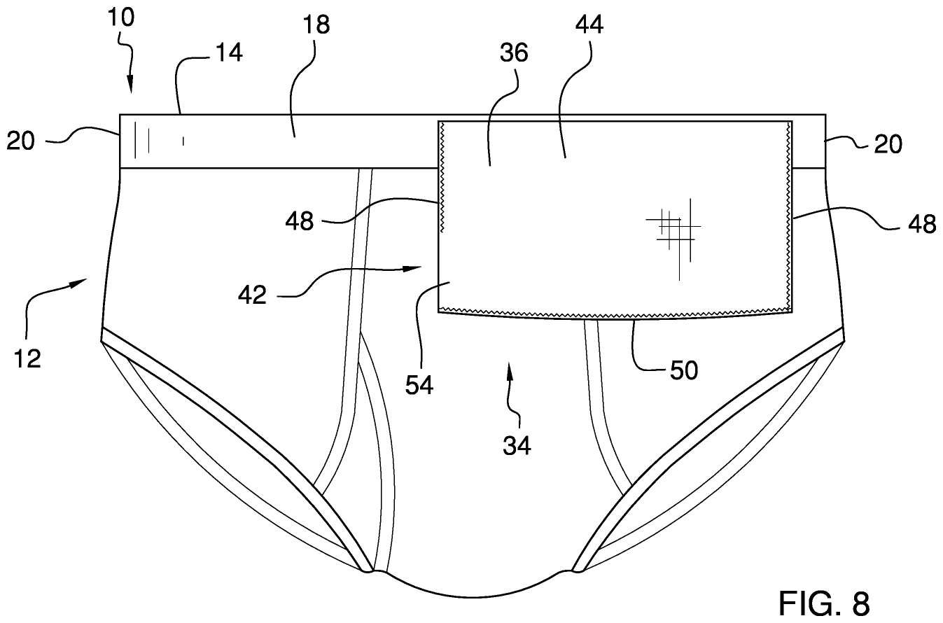

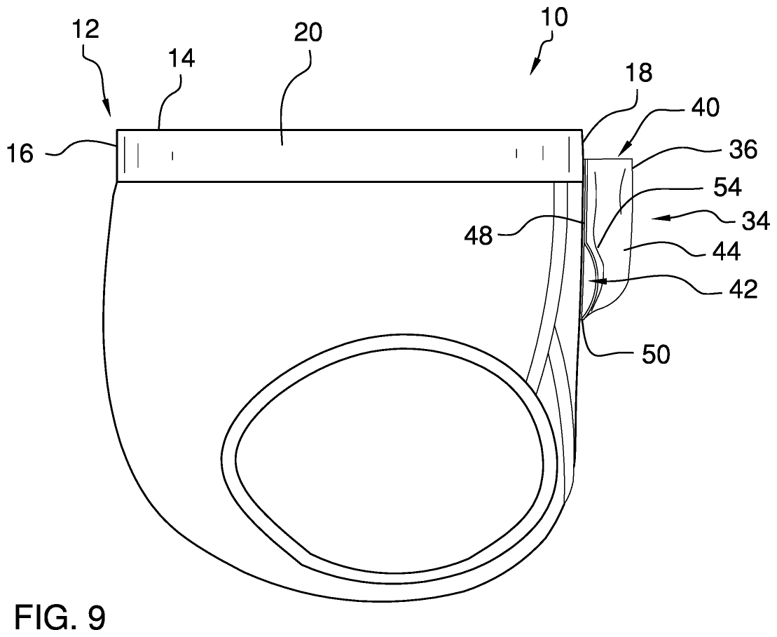

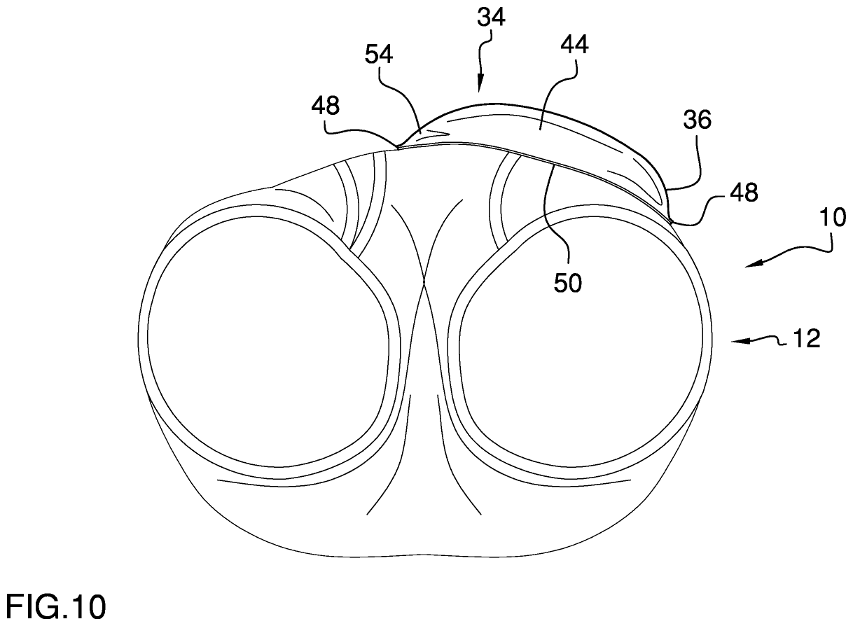

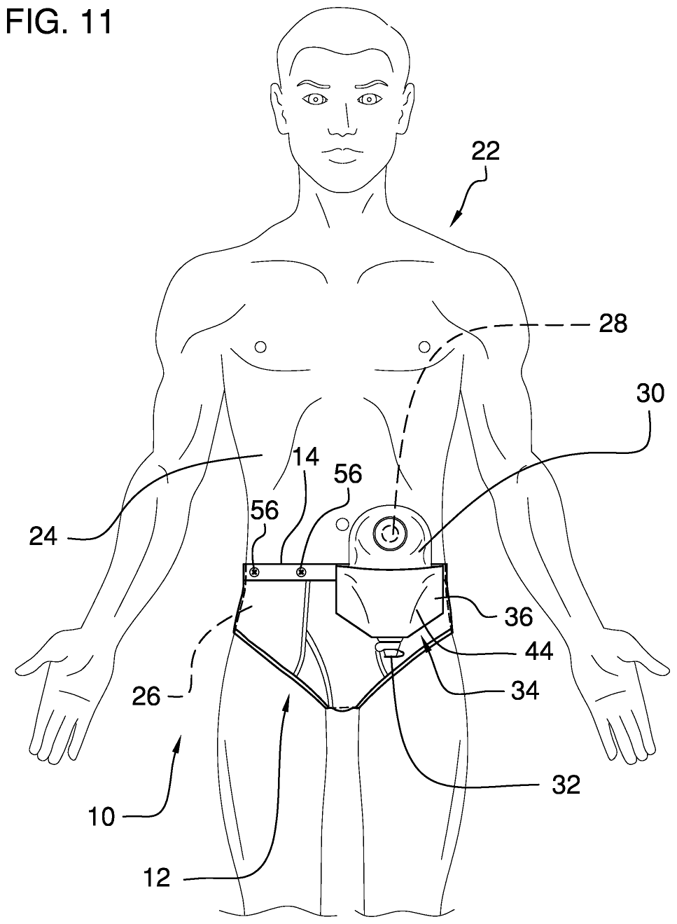

With reference now to the drawings, and in particular to FIGS. 1 through 11 thereof, a new ostomy bag holding garment embodying the principles and concepts of an embodiment of the disclosure and generally designated by the reference numeral 10 will be described.

As best illustrated in FIGS. 1 through 11, the clothing article 10 generally comprises an undergarment 12 which is configured to be worn over a lower torso 26 and genitals of a user 22. The undergarment 12 has a waistband 14 which extends around a torso 24 of the user 22 when the undergarment 12 is worn by the user 22. A pouch 34 is mounted to an outer surface 16 of the waistband 14 and is configured for holding a ostomy bag 30. The pouch 34 comprises a wall 36 bounding a cavity 38 for at least partially containing the ostomy bag 30, and the pouch 34 is removably attached to the waistband 14. The pouch 34 is also selectively positionable in one of a plurality of locations along the front side 18 of the waistband 14 between a pair of lateral sides 20 of the waistband 14.

The pouch 34 has an open top 40 and has an aperture 42 which extends through the wall 36 to the cavity 38. The aperture 42 has a size such that the aperture 42 is configured for receiving a vent portion 32 of the ostomy bag 30 therethrough. The aperture 42 is positioned such that the vent portion 32 of the ostomy bag 30 is positioned below a stoma 28 of the user 22 when the undergarment 12 is worn by the user 22. The wall 36 comprises a resiliently stretchable material such that the wall 36 stretches to accommodate an expansion of the ostomy bag 30 caused by the ostomy bag 30 filling with waste. Depending on the type of ostomy (e.g., colostomy, ileostomy, urostomy, etc.) the waste may comprise feces, urine, or the like. The wall 36 also comprises a front panel 44 and a rear panel 46, in which the front and rear panels 44, 46 are attached to each other along associated lateral edges 48 and bottom edges 50 thereof. The open top 40 extends between associated upper edges 52 of the front and rear panels 44, 46, and the aperture 42 extends between the associated bottom edges 50 of the front and rear panels 44, 46. The associated bottom edges 50 of the front and rear panels 44, 46 are angled downwardly toward the aperture 42 from one of the associated lateral edges 48 of the front and rear panels 44, 46.

In some embodiments, the pouch 34 may be fixedly attached to the waistband 14. Also in some embodiments, the aperture 42 may also be positioned on a lateral portion 54 of the pouch 34 to direct the vent portion 32 of the ostomy bag 30 in a lateral direction in cases where such an orientation is more comfortable or otherwise advantageous to the user 22.

A plurality of first mating members 56 and a plurality of second mating members 58 are provided, wherein the plurality of first mating members 56 is coupled to the front side 18 of the waistband 14 of the undergarment 12 and the plurality of second mating members 58 is coupled to the wall 36 of the pouch 34. At least a portion of the plurality of first mating members 56 being coupled to at least a portion of the plurality of second mating members 58 when the pouch 34 is positioned in each location of the plurality of locations. The first and second mating members 56, 58 are buttons and holes respectively, but the first and second mating members 56, 58 may comprise snaps, hook-and-eye connectors, hook-and-loop fasteners, or the like.

In use, the ostomy bag 30 is positioned in the pouch 34 so that it is supported by the undergarment 12 and held away from the skin of the user 22. The pouch 34 is positioned along the waistband 14 of the undergarment 12 in a selected position to most comfortably position the ostomy bag 30. The vent portion 32 may be opened while the ostomy bag 30 is held in the pouch 34 to release the waste from within the ostomy bag 30.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A clothing article for holding an ostomy bag while the ostomy bag is fluidly coupled to a stoma in a torso of a user, the clothing article comprising:
an undergarment being configured to be worn over a lower torso and genitals of the user, the undergarment having a waistband which extends around the torso of the user when the undergarment is worn by the user; and
a pouch being mounted to an outer surface of the waistband and being configured for holding the ostomy bag, the pouch comprising a wall bounding a cavity for at least partially containing the ostomy bag, the pouch being removably attached to the waistband.

2. The clothing article of claim 1, wherein the pouch is selectively positionable in one of a plurality of locations along the front side of the waistband between a pair of lateral sides of the waistband.

3. The clothing article of claim 2, further comprising a plurality of first mating members and a plurality of second mating members, the plurality of first mating members being coupled to the front side of the waistband of the undergarment, the plurality of second mating members being coupled to the wall of the pouch, at least a portion of the plurality of first mating members being coupled to at least a portion of the plurality of second mating members when the pouch is positioned in each location of the plurality of locations.

4. The clothing article of claim 1, wherein the pouch has an open top.

5. The clothing article of claim 1, wherein the pouch has an aperture extending through the wall to the cavity, the aperture having a size such that the aperture is configured for receiving a vent portion of the ostomy bag therethrough.

6. The clothing article of claim 1, wherein the aperture is positioned such that the vent portion of the ostomy bag is positioned below the stoma when the undergarment is worn by the user.

7. The clothing article of claim 1, wherein the wall comprises a resiliently stretchable material such that the wall stretches to accommodate an expansion of the ostomy bag caused by the ostomy bag filling with waste.

8. The clothing article of claim 1, wherein the wall comprises a front panel and a rear panel, the front and rear panels being attached to each other along associated lateral edges and bottom edges thereof.

9. The clothing article of claim 10, wherein the open top extends between associated upper edges of the front and rear panels.

10. The clothing article of claim 10, wherein the aperture extends between the associated bottom edges of the front and rear panels.

11. The clothing article of claim 12, wherein the associated bottom edges of the front and rear panels are angled downwardly toward the aperture from one of the associated lateral edges of the front and rear panels.

12. A clothing article for holding an ostomy bag while the ostomy bag is fluidly coupled to a stoma in a torso of a user, the clothing article comprising:
an undergarment being configured to be worn over a lower torso and genitals of the user, the undergarment having a waistband which extends around the torso of the user when the undergarment is worn by the user;
a pouch being mounted to an outer surface of the waistband and being configured for holding the ostomy bag, the pouch comprising a wall bounding a cavity for at least partially containing the ostomy bag, the pouch being removably attached to the waistband, the pouch being selectively positionable in one of a plurality of locations along the front side of the waistband between a pair of lateral sides of the waistband, the pouch having an open top, the pouch having an aperture extending through the wall to the cavity, the aperture having a size such that the aperture is configured for receiving a vent portion of the ostomy bag therethrough, the aperture being positioned such that the vent portion of the ostomy bag is positioned below the stoma when the undergarment is worn by the user, the wall comprising a resiliently stretchable material such that the wall stretches to accommodate an expansion of the ostomy bag caused by the ostomy bag filling with waste, the wall comprising a front panel and a rear panel, the front and rear panels being attached to each other along associated lateral edges and bottom edges thereof, the open top extending between associated upper edges of the front and rear panels, the aperture extending between the associated bottom edges of the front and rear panels, the associated bottom edges of the front and rear panels being angled downwardly toward the aperture from one of the associated lateral edges of the front and rear panels; and
a plurality of first mating members and a plurality of second mating members, the plurality of first mating members being coupled to the front side of the waistband of the undergarment, the plurality of second mating members being coupled to the wall of the pouch, at least a portion of the plurality of first mating members being coupled to at least a portion of the plurality of second mating members when the pouch is positioned in each location of the plurality of locations.
